(19) United States
(12) Patent Application Publication
(10) Pub. No.: US 2012/0283224 A1
(43) Pub. Date: Nov. 8, 2012

(54) CHEMICAL STRUCTURE COMPRISING PHOSPHATES WITH POLYOL SUBSTRUCTURES FOR HAIR AND SKIN COSMETICS USAGE

(75) Inventors: Maria Del Carmen Velazquez Pereda, Sao Paulo (BR); Marcio Antonio Polezel, Indaiatuba (BR); Gustavo De Campos Dieamant, Sorocaba (BR); Cecília Nogueira, Sorocaba (BR); Carlos Roque Duarte Correia, Campinas (BR); Nilton Soares Camilo, Campinas (BR); Jéssica Eleonora Pedroso Sanches Silveira, Sorocaba (BR)

(73) Assignee: CHEMYUNION QUIMICA LTDA, Sorocaba (BR)

(21) Appl. No.: 13/505,924

(22) PCT Filed: Nov. 3, 2009
(86) PCT No.: PCT/IB09/54883

§ 371 Date: Jul. 16, 2012

(51) Int. Cl. C07F 9/09; A61K 31/661; A61P 17/08; A61Q 19/00; A61P 17/00; A61K 8/55; A61P 17/10

(52) U.S. Cl. 514/129; 558/177

(57) ABSTRACT

A composition suitable for application to hair and/or skin comprises Phosphates Esters, such as Xylitol Phosphate Esters and other Polyol Units Phosphoric Acid Esters. This composition can be used to improve or treat skin, including scalp, conditions in cosmetic or pharmaceutical products for acne, dandruff, seborrheic dermatitis and malodor and/or can be incorporate in such formulations as a coemulsifying agent or a preservative, alone or in combination to other emulsifying or preservative agents in order to increase such activities.

### FIELD OF INVENTION

The invention relates to chemical structures suitable for topical application to hair and/or skin. In particular, the invention is concerned to chemical structures suitable for reducing dandruff, acnes vulgaris and for deodorant products and acting as a coemulsifier or preservative agent in cosmetic and pharmaceutical products.

### BACKGROUND OF INVENTION AND PRIOR ART

There are several skin conditions that cause individuals discomfort such as acne, dandruff, seborrheic dermatitis and body perspiration odor, so that effective treatment is necessary.

Acne vulgaris is manifested by comedones, papules, pustules, and cysts. The etiology of acne appears to be multifactorial. The exact mechanism triggering the development of the comedone and the stimuli causing the non-inflamed lesion to become inflamed are poorly understood. The microbiology of acne vulgaris and its immunologic ramifications constitute the major thrust of present research in the elucidation of the pathogenesis of the inflammatory acne lesion.

The microorganisms that may be involved in this process are Propionibacterium, Staphylococci and Mycrococci and Malassezia.

The pathophysiology of acne involves overproduction of sebum, abnormal desquamation of sebaceous follicle epithelium and proliferation of Propionibacterium acnes bacteria, which generate an inflammatory response. Comedogenesis, characterized by abnormal desquamation of follicular corneocytes into the sebaceous follicle duct, is primary in the developmental process of acne.

Its treatment considerations include correcting the altered pattern of follicular keratinization, reducing sebaceous gland production, diminishing the Propionibacterium acnes population in the follicle and inhibiting its production of extracellular inflammatory products, and producing an anti-inflammatory effect.

This disorder has been treated primarily by topically applying keratolytic agents such as benzoylperoxide, antibacterial compounds, or combinations thereof such as benzoylperoxide and miconazole. Retinols and retinoids may be used, but undesirable effects, such as skin irritation.

However, products which not only treat the disorder but also improve skin condition without causing side effects are needed.

U.S. Pat. No. 5,449,519 describes cosmetic compositions having keratolytic and anti-acne activity.

U.S. Pat. No. 5,976,521 describes an anti-acne cosmetic composition which presents moisturization benefits in the form of multiple phase water-in-oil emulsion and process for preparation thereof.

U.S. Patent 2009/0214628 A1 comprises a cosmetic composition for treating human skin, ameliorating skin conditions, such as acne and dandruff.

Dry scalp flaking, dandruff and seborrheic dermatitis are chronic scalp manifestations of similar etiology differing only in severity. The common etiology is a convergence of three factors: sebaceous gland secretions, microfloral metabolism and individual susceptibility.

Dandruff is a common complaint and is suffered by as many as 50% of the population at some time during life. It is characterized by flaking, crusting, erythema, scaling, itching and hair breakage and the probable causal agent of dandruff is Malassezia, previously known as Pytyrosporum, so that the most effective antidandruff treatments are antifungals.

It was also demonstrated that scalp skin in dandruff sufferers contains reduced levels of intercellular lipids and shows an increased response to histamine. As the skin barrier is impaired, dandruff sufferers are more prone to irritant adverse effects of microbial activity.

Since sebum and scalp scale are well-known requirements for the initiation of dandruff, traditional antidandruff shampoos usually focus on delivering active anti-dandruff ingredients and through removal of scale and sebum.

Patent WO/2009/080306 relates to the use of peptides as active ingredients for the treatment or inhibition of dandruff while U.S. Pat. No. 7,547,752 relates to a composition for treating the scalp which comprises one anti-dandruff agent, conjugated linoleic acid and a carrier.

The marketing of products specifically made for controlling underarm wetness and odor only began slightly over 100 years ago.

Sweating is the elaboration of a fluid secretion on the general body surface produced by sweat glands within the skin.

Thermoregulatory sweating is clearly of selective advantage since it permits work to be done under conditions otherwise adverse to heat dissipation, that is, above the thermoneutral zone.

The control of sweating in mammals appears to differ depending on the nature of the stimulus. In man, although mental activity can induce a response, the two main triggers are heat and exercise.

It has been established that heat-induced sweating requires an intact sympathetic nervous system while that induced by exercise also involves humoral stimulation by circulating catecholamines from the adrenal medulla.

Research on the formation and control of axillary malodors has led to the identification of steroids and acids which represent this unique body odor. The characterization of the secretions of the underarm and the skin bacteria involved in odor formation has resulted in a comprehensive picture of the environment of the underarm. Standard odor control mechanisms such as use of antimicrobials and antiperspirant salts to reduce bacterial action or substantive fragrances to mask odors are still the commercial methods for reducing body odor.

U.S. Pat. No. 5,958,975 comprises compositions containing an aryl 2-acetoxyethanoic acid for the therapeutic treatment as well as prophylactic measures by topical application to eradicate or prevent the development of axillary foul odor, foot malodor and other body odor.

U.S. Pat. No. 5,660,838 describes xylobiose-containing skin preparations for external use, not only in preventing the occurrence of stickiness, color change and malodor in the skin preparations but also in providing enhanced moisture retention and reducing excessive roughness and dryness of the skin and hair.

U.S. Pat. No. 5,874,067 encompasses a method of controlling malodors on human skin comprising the application to the human skin of a composition comprising solubilized, water-soluble, uncomplexed cyclodextrin which can be applied directly as a spray, poured from a bottle and applied by hand, or applied via a wipe.

Cosmetics, toilet, perfume and cleaning products, as well as medicines and other pharmaceutical consumables, shall be duly conserved for withstanding the processes of manufacture, storage, handling by the consumer and consequent maintenance of the health integrity of the user.

The conserving agents shall be bioactive molecules effectively acting with the destruction or inhibition of microorganism growth. For this reason, the action spectrum and the used concentration shall be accurately calculated to assure the integrity of the product. The ideal conserving agent shall be stable, compatible with the other ingredients of the formulation, have a with action specter in low concentration and be active in a large range of pH, besides not causing toxic, irritating and hypersensitizing effects. However, few agents approximate this ideal, and all, without exceptions, have demonstrated causing contact sensitizing.

Preservatives which are currently used in cosmetics are parabens, phenoxyethanol, formaldehydes and releasers of formaldehyde, potassium sorbate and 3-iodo-2-propynyl-butylcarbamate (IPBC).

Considering that the conserving agents available in the market present, without exceptions, some risk of hypersensitivity, efforts have been directed to developing natural compounds with antimicrobial activity with the purpose of reducing or even replacing the traditional conserving agent.

U.S. Pat. No. 6,447,793 comprises water soluble, broad spectrum preservative system for cosmetic and personal care products and U.S. Pat. No. 4,966,754 Patent describes essential oils as preservative for cosmetic compositions.

Some molecules are being developed combining phosphoric acid ester to be used in cosmetic, pharmaceutical and dermatological compositions.

U.S. Pat. No. 5,139,781 describes compositions for topical applications that comprise monoalkyl or monoalkenyl phosphate surfactant for topical application intended primarily as a personal washing product.

Patent PCT/IB2008/054321, applicant Chemyunion Química Ltda., comprises xylitol esters and ethers applied as alternative emulsifiers, solvents, coemulsifiers and preservative systems for pharmaceutical and cosmetic products.

Patent PCT/EP2008/006220 from Clariant International Ltd. comprises phosphoric acid esters containing phosphorus atoms bridged by polyol units.

### BRIEF DESCRIPTION OF THE FIGURES

FIG. 1—The oil reduction related to a product for acne was expressed in sebumetric index values which were measured on the first day of the study before product application (D1/T0) and on the last day of the study after 28 days of use (D28).

FIG. 2—The study participants' opinion concerning a product for decreasing acne was expressed in percentage of satisfied subjects according to each assessed characteristic (improvement in skin oiliness, reduction of comedones/blackheads, and reduction of inflammatory comedones/pimples, decrease of inflammation and increase of moisturization).

FIG. 3—The oil reduction related to a product for dandruff was expressed in sebumetric index values which were measured on the first day of the study before product application (D1/T0) and on the last day of the study after 28 days of use (D28).

FIG. 4—The study participants' opinion concerning a product for ameliorating dandruff was expressed in percentage of satisfied subjects according to each assessed characteristic (improvement in skin oiliness, improvement in hair oiliness, reduction of dandruff, reduction of dandruff fall on the shoulders, improvement of pruritus, and reduction of hair fall).

FIG. 5—Odor variation was graded according to a score scale from 1 to 10 after 8 h from the product application for 2 products, Xylityl Phosphate Esters which was a composition containing 3% of this ingredient and Triclosan which was a preparation containing 0.2% of this substance.

FIG. 6—Odor variation was graded according to a score scale from 1 to 10 after 24 h from the product application for 2 products, Xylityl Phosphate Esters which was a composition containing 3% of this ingredient and Triclosan which was a preparation containing 0.2% of this substance.

### DEFINITION OF INVENTION

Accordingly, the invention provides a chemical structures suitable for topical application to the skin or hair, particularly for the treatment of skin and scalp, which comprises Phosphates, Phosphonates.

Regarding the use of the invention in skin including the scalp, the chemical structures are useful for alleviating the symptoms of cosmetic and dermatological conditions such as acne, dandruff, seborrheic dermatitis and odor changing.

This patent application aims to the incorporation/utilization of Phosphates obtained from organic synthesis in cosmetic and pharmaceutical compositions suitable for application to skin or hair, especially those for decreasing acne, dandruff, seborrheic dermatitis and odor changes resulting from perspiration.

These chemical structures can present preservative action, as well as against microorganisms, such as Propionibacterium and Malassezia and also can be used as a coemulsifying agent.

### DISCLOSURE OF INVENTION

This invention comprises Phosphates, having the following structure:

### Structure

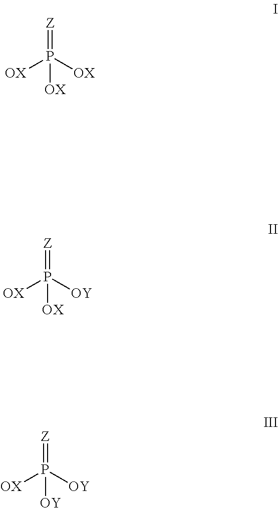

### Where:

a) OX=xylitol, sorbitol, glycerol and mannitol.
b) Y=H, alkali metal, ammonium, substituted ammonium counter-ions or is a linear or branched, saturated alkyl group having 4 to 30, preferably 8 to 22 and more preferably 12 to 18 carbon atoms, or a linear or branched, mono-or polyunsaturated alkenyl group with 4 to 30, preferably 8 to 22 and particularly preferably 12 to 18 carbon atoms.

### 1. Clinical Assessment of the Skin Oiliness of A Product For Face Care

The skin oiliness was assessed in 32 subjects with oily/combination skin, acne grade I or II and 12 to 45 years old who has used a facial cosmetic product containing 3% of Xylityl Phosphate Esters twice a day.

The skin oiliness was measured on the face of 15 subjects with Sebumeter® SM810 PC equipment (Courage+Khazaka). The measurements were taken on the first day of the study before the product application and after 28 days of product use.

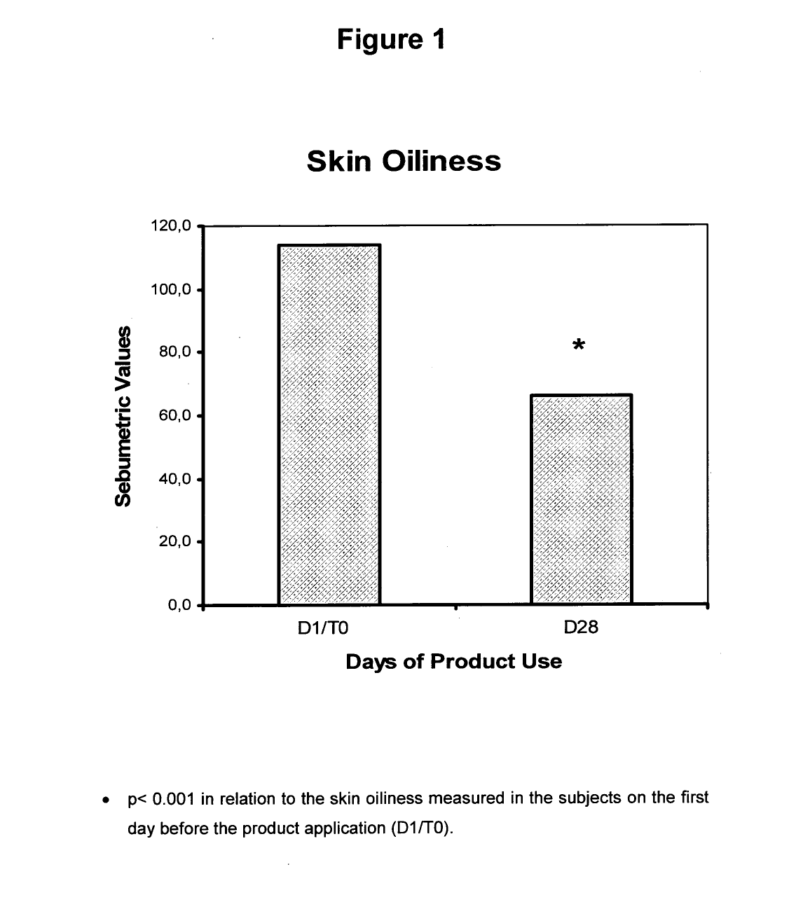

The results were expressed in absolute values of sebumetric index, as the transparency of a plastic tape which correlates to skin sebum amount is measured. The oiliness reduction was analyzed through t-test statistical analysis (FIG. 1).

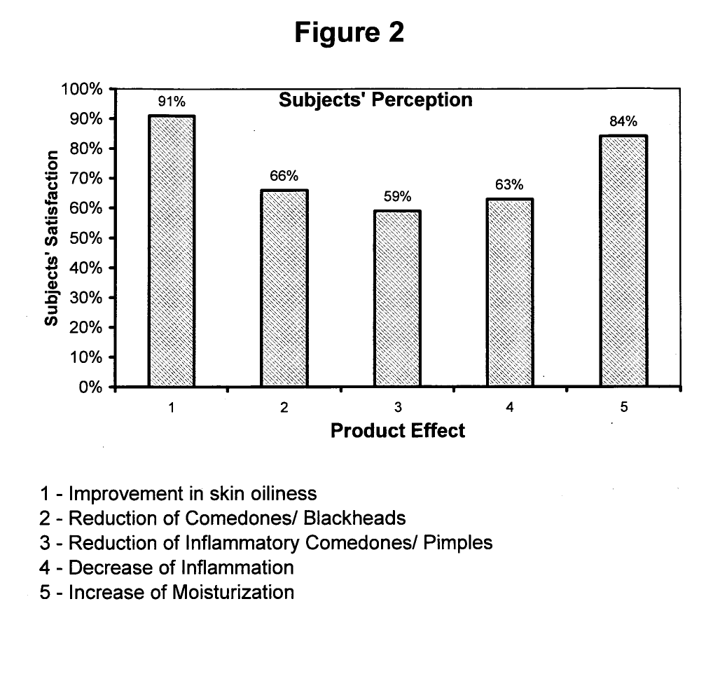

All the subjects were asked to answer a questionnaire about their perception regarding the 28 days of product use. The questions were related to improvement of skin oiliness, reduction of comedones/blackheads, reduction of inflammatory comedones/pimples, decrease of inflammation and increase of moisturization. The results were presented in percentage of satisfied subjects (FIG. 2).

FACIAL TONIC LOTION

(% w/w)

PHASE A

| | |
|---|---|
| Water q.s | 100.00 |
| Xylityl Phosphate Esters | 3.00 |
| Glycerin | 3.00 |
| Ethyl Alcohol 96% | 15.00 |

PHASE B

| | |
|---|---|
| Sodium Hydroxide 20% q.s | pH = 6.0-7.0 |

PHASE C

| | |
|---|---|
| DMDM Hydantoin (and) lodopropynyl Butylcarbamate | 0.30 |

### 2. Clinical Assessment of the Skin Oiliness of Two Products For Hair Care

The skin oiliness was assessed in 31 subjects with dandruff or story of dandruff/seborrhea and 18 to 60 years old who has used a shampoo and a hair lotion containing 3% of Xylityl Phosphate Esters each once a day. The skin oiliness was measured on the frontal region of the scalp of 15 subjects with Sebumeter® SM810 PC equipment (Courage+Khazaka). The measurements were taken on the first day of the study before the product application and after 28 days of product use.

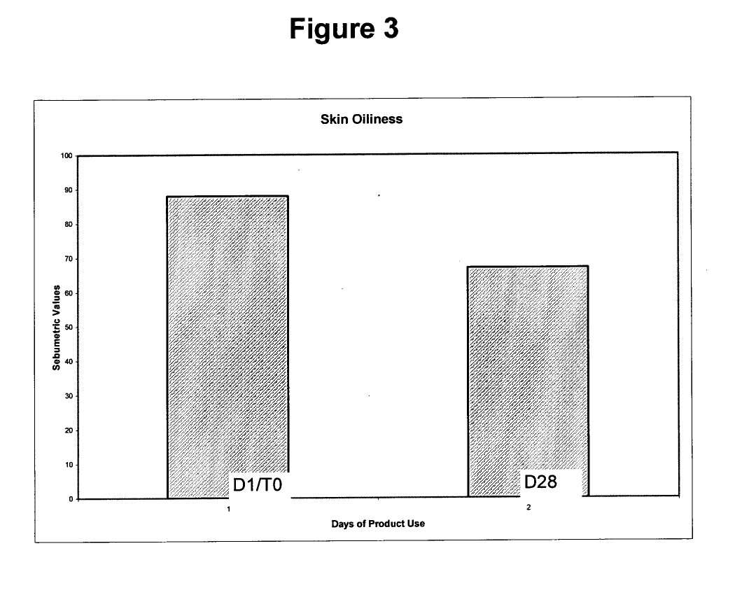

The results were expressed in absolute values of sebumetric index, as the transparency of a plastic tape which correlates to skin sebum amount is measured. The oiliness reduction was analyzed through t-test statistical analysis (FIG. 3).

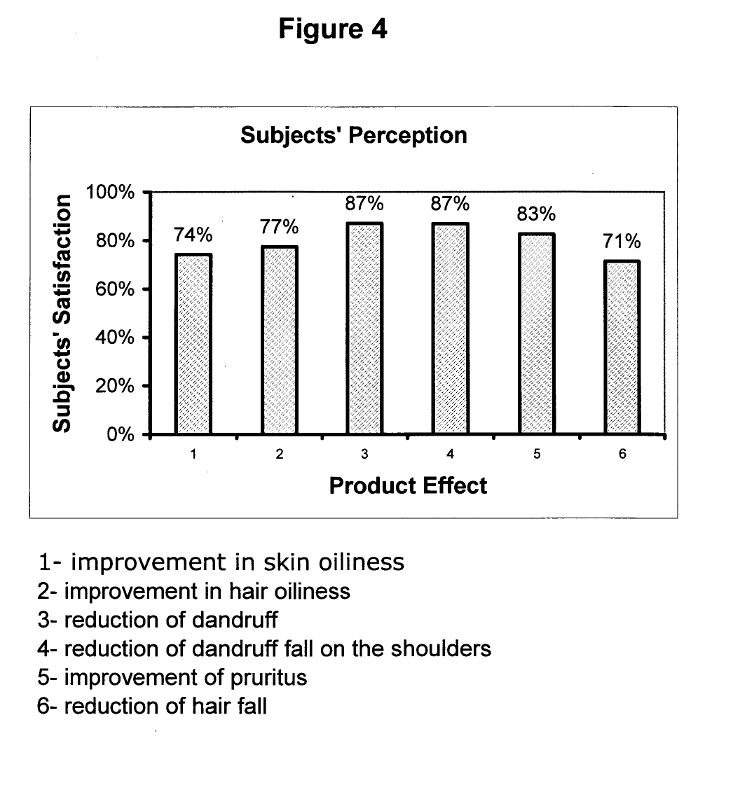

All the subjects were asked to answer a questionnaire about their perception regarding the 28 days of products use. The questions were related to improvement in skin oiliness, improvement in hair oiliness, reduction of dandruff, reduction of dandruff fall on the shoulders (considering the subjects that have reported this event), improvement of pruritus (considering the subjects that have reported this event) and reduction of hair fall (considering the subjects that have reported this event). The results were presented in percentage of satisfied subjects (FIG. 4).

SHAMPOO

(% w/w)

PHASE A

| | |
|---|---|
| Water qs | 100.00 |
| Disodium EDTA | 0.10 |
| Cocamidopropyl Betaine 30% | 4.00 |
| Sodium Lauryl Ether Sulfate 27% | 30.00 |
| Polyquaternium-7 | 0.50 |

PHASE B

| | |
|---|---|
| Decyl Glucoside | 2.00 |
| PEG 120 Methyl Glucose Dioleate | 1.50 |
| Phenoxyethanol (and) Ethylparaben (and) Methylparaben | 0.50 |
| (and) Propylparaben (and) Butylparaben | |

PHASE C

| | |
|---|---|
| Sodium Hydroxide 20% q.s | pH = 6.5-7.0 |

PHASE D

| | |
|---|---|
| Water | 10.00 |
| Xylityl Phosphate Esters | 3.00 |

PHASE E

| | |
|---|---|
| Sodium Cloride | q.s. |

HAIR TONIC LOTION

(% w/w)

PHASE A

| | |
|---|---|
| Water qs | 100.00 |
| Xylityl Phosphate Esters | 3.00 |
| Glycerin | 2.00 |
| Ethyl Alcohol 96% | 20.00 |

PHASE B

| | |
|---|---|
| Sodium Hydroxide 20% q.s | pH = 6.0-7.0 |

PHASE C

| | |
|---|---|
| DMDM Hydantoin (and) lodopropynyl Butylcarbamate | 0.30 |

### 3. Clinical Assessment of the Deodorant Efficacy of A Cosmetic Product

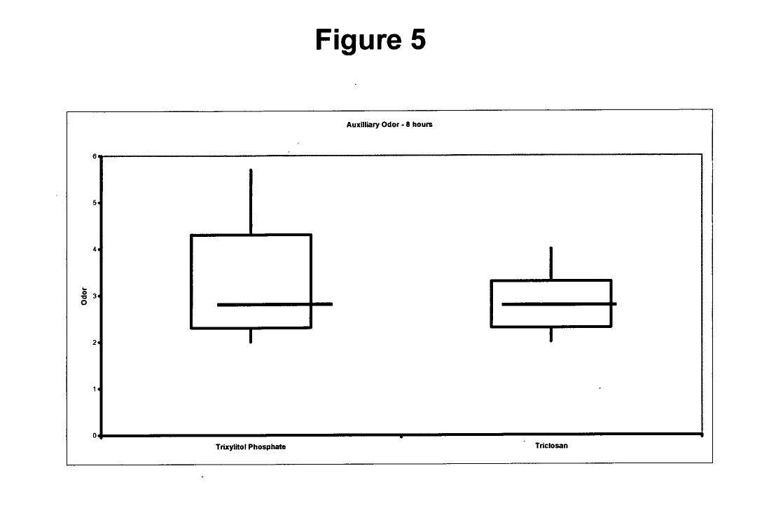

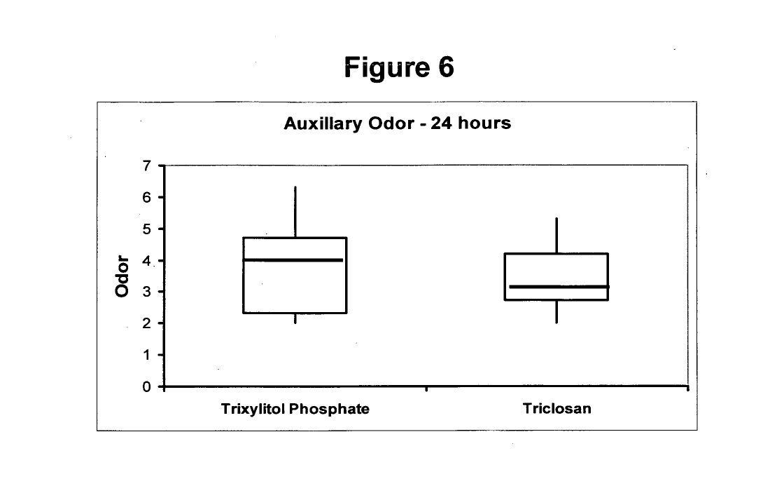

The deodorant effect was assessed in 14 subjects and a cosmetic product containing 3% of Xylityl Phosphate Esters was compared to a product containing 0.2% of Triclosan. The products were applied in the axillary area for 3 days after a period of 21 days without using antiperspirants. The auxiliary odor was assessed 8 and 24 hours after the last application of the test products. The odor intensity was assessed by trained technicians who gave grades according to a score scale from 0—None/Absence of malodor to 10—Extremely strong malodor. The reduction of malodor was analyzed through Student t-test statistical analysis (FIGS. 5 and 6). As no statistical difference was presented, it can be suggested that the products presented similar behavior related to the observed parameters.

DEODORANT

(% w/w)

PHASE A

| | |
|---|---|
| Ethyl Alcohol 96% | 40.00 |

PHASE B

| | |
|---|---|
| Water | 57.00 |
| Xylitol Phosphate Esters | 3.00 |

1.-11. (canceled)

12. Phosphate esters obtained by polyol units involving xylitol, sorbitol, glycerol and mannitol comprising chemical structures represented by mono, di and tri-esters.

13. Cosmetic formulation, in particular as liquid tonics, semisolid formulation, emulsions, liquid soap and dermocosmetics formulations, characterized in that it contains an ester according to claim 12 in an amount of 1-10%.

14. Topical formulations containing chemical structures according to claim 12 for acne, dandruff or oily skin treatment.

15. Topical formulations containing chemical structures according to claim 12 for seborrheic dermatitis treatment.

16. Topical formulations containing chemical structures according to claim 12 for deodorant formulations.
